(19) United States
(12) Patent Application Publication
(10) Pub. No.: US 2025/0332015 A1
(43) Pub. Date: Oct. 30, 2025

(54) HAPTICALLY-ENABLED FOOT ANKLE ORTHOSIS FOR POST-INJURY PLANAR PRESSURE MONITORING AND MODULATION

(71) Applicants: Georgia Tech Research Corporation, Atlanta, GA (US); Emory University, Atlanta, GA (US)

(72) Inventors: Frank L. Hammond, Atlanta, GA (US); Jason Bariteau, Atlanta, GA (US); Ana Sanz Cozcolluela, Atlanta, GA (US); Joseph Colin Novack, Atlanta, GA (US); Cassandra Ashleigh Tom, Atlanta, GA (US)

(21) Appl. No.: 18/869,731

(22) PCT Filed: May 26, 2023
(86) PCT No.: PCT/US2023/067541

§ 371 Date: Nov. 26, 2024

Related U.S. Application Data
(60) Provisional application No. 63/365,443, filed on May 27, 2022.

(51) Int. Cl. A61F 5/01

(52) U.S. Cl. CPC A61F 5/0111; A61F 2005/0155

(57) ABSTRACT

An exemplary embodiment of the present disclosure provides a haptic feedback device, comprising one or more pressure sensors and a haptic feedback sleeve. The one or more pressure sensors can be configured to measure a pressure applied to the foot of a user. The haptic feedback sleeve can be configured to provide haptic feedback to the user. The haptic feedback can be based, at least in part, on the pressure applied to the foot of the user.

### CROSS-REFERENCE TO RELATED APPLICATIONS

This application claims the benefit of U.S. Provisional Application Ser. No. 63/365,443, filed on 27 May 2022, which is incorporated herein by reference in its entirety as if fully set forth below.

### FIELD OF THE DISCLOSURE

The various embodiments of the present disclosure relate generally to orthoses, and more particularly to foot-ankle orthoses.

### BACKGROUND

Ankle sprains are one of the most common orthopedic injuries in the U.S., affecting as many as two million people each year. These sprains can range from a mild grade, which involves ligament strains and/or incomplete tears but allows the patient to weight-bear immediately, to a severe (acute) grade the involves complete ligament ruptures and can require surgery, prolonged periods of immobilization, and possible limitations on weightbearing. After mild sprains, patients often develop an abnormal gait pattern and walk on the lateral border of the foot to reduce stress and pain. This uneven distribution of pressures on the plantar surface of the foot can slow the rehabilitation process and increase the incidence of reinjury due to inadvertent ankle rolling, even when orthotic braces or wraps are used. After surgery for acute ankle sprains, patients must avoid bearing any weight on the affected foot until the joint is stable, and then must gradually increase the weight they bear until they reach a normal, healthy load-bearing level.

Currently, there is a dearth of methods for monitoring plantar surface pressures outside the clinic and guiding patients to correct their gait mechanics or weight bearing tendencies and avoid exacerbating their injury. There are also few ways to provide orthopedic specialists with detailed, accurate data on their patients' gait mechanics so that postoperative rehabilitation protocols can be updated regularly, and recovery times optimized. Accordingly, there is a need for devices and methods that address these deficiencies.

### BRIEF SUMMARY

An exemplary embodiment of the present disclosure provides a haptic feedback device, comprising one or more pressure sensors and a haptic feedback sleeve. The one or more pressure sensors can be configured to measure a pressure applied to the foot of a user. The haptic feedback sleeve can be configured to provide haptic feedback to the user. The haptic feedback can be based, at least in part, on the pressure applied to the foot of the user.

In any of the embodiments disclosed herein, the one or more pressure sensors can be configured to measure pressure applied to one or more of the calcaneus bone, hallux, cuboid, and medial aspect of the user's foot.

In any of the embodiments disclosed herein, the one or more pressure sensors can comprise: a first pressure sensor configured to measure pressure applied to a calcaneus bone of the user's foot; a second pressure sensor configured to measure pressure applied to a hallux of the user's foot; a third pressure sensor configured to measure pressure applied to a cuboid of the user's foot; and a fourth pressure sensor configured to measure pressure applied to a medial aspect of the user's foot.

In any of the embodiments disclosed herein, the one or more pressure sensors can be force sensitive resistors.

In any of the embodiments disclosed herein, the haptic sleeve can comprise one or more vibrotactile cells configured to apply a stimulus to the user.

In any of the embodiments disclosed herein, the one or more vibrotactile cells can be configured to apply a stimulus to at least a portion of the leg of the user.

In any of the embodiments disclosed herein, the one or more vibrotactile cells can comprise: a first vibrotactile cell configured to apply a stimulus to an anterior aspect of a leg of the user; a second vibrotactile cell configured to apply a stimulus to a posterior aspect of a leg of the user; a third vibrotactile cell configured to apply a stimulus to a medial aspect of a leg of the user; and a fourth vibrotactile cell configured to apply a stimulus to a lateral aspect of a leg of the user.

In any of the embodiments disclosed herein, two or more of the one or more vibrotactile cells are configured to provide simultaneous stimuli to the user.

In any of the embodiments disclosed herein, two or more of the one or more vibrotactile cells can be configured to provide sequential stimuli to the user.

In any of the embodiments disclosed herein, can further comprise one or more microcontrollers configured to collect measurements taken by the one or more pressure sensors and control the haptic feedback provided to the user.

In any of the embodiments disclosed herein, the one or more microcontrollers can be further configured to control one or more of a frequency, amplitude, or pattern of the haptic feedback provided to the user.

In any of the embodiments disclosed herein, the haptic feedback can be further based, at least in part, on a predetermined pressure threshold.

In any of the embodiments disclosed herein, the one or more pressure sensors can be configured to wirelessly transmit pressure measurements to the haptic feedback sleeve.

In any of the embodiments disclosed herein, the one or more pressure sensors can be configured to transmit pressure measurements to the haptic feedback sleeve via a wired connection.

In any of the embodiments disclosed herein, the haptic feedback sleeve can comprise one or more straps to secure the sleeve to the user.

In any of the embodiments disclosed herein, the haptic feedback sleeve can comprise an elastic material configured to provide a compressive force to the user.

In any of the embodiments disclosed herein, the haptic feedback sleeve can be configured to be secured to the calf of the user.

In any of the embodiments disclosed herein, the haptic feedback sleeve can be configured to be secured to the thigh of the user.

In any of the embodiments disclosed herein, the one or more pressure sensors can be positioned on a shoe insert.

In any of the embodiments disclosed herein, the haptic feedback device can further comprise an accelerometer configured to determine when the user is moving.

Another embodiment of the present disclosure provides a method of providing haptic feedback to a user. The method can include applying one or more pressure sensors configured to measure a pressure applied to a foot of the user, measuring one or more pressures applied to the foot, and applying, to the user, one or more stimuli based, at least in part, on the measured pressure.

In any of the embodiments disclosed herein, measuring one or more pressures applied to the foot can include one or more of measuring a first pressure applied to a calcaneus bone of the user's foot, measuring a second pressure applied to a hallux of the user's foot, measuring a third pressure applied to a cuboid of the user's foot, and measuring a fourth pressure applied to a medial aspect of the user's foot.

In any of the embodiments disclosed herein, applying to the user one or more stimuli can include one or more of applying a first stimulus to an anterior aspect of a leg of the user upon the first pressure exceeding a first minimum threshold, applying a second stimulus to a posterior aspect of a leg of the user upon the second pressure exceeding a second minimum threshold, applying a third stimulus to a medial aspect of a leg of the user upon the third pressure exceeding a third minimum threshold, and applying a fourth stimulus to a lateral aspect of a leg of the user upon the fourth pressure exceeding a fourth minimum threshold.

In any of the embodiments disclosed herein, the intensity of one or more of the first, second, third, and fourth stimuli can vary proportionally to the first, second, third, and/or fourth pressures, respectively.

In any of the embodiments disclosed herein, the method can further include continuously recording one or more of the first, second, third, and fourth pressures during an exercise of the user, comparing the recorded pressures to at least one corresponding predetermined injury threshold, and increasing the intensity of each of the first, second, third, and fourth stimuli when the recorded pressures exceed the at least one corresponding predetermined injury threshold.

In any of the embodiments disclosed herein, the one or more stimuli can comprise vibrotactile feedback.

In any of the embodiments disclosed herein, the method can further include scaling the intensity of the first stimulus based on a sensitivity of the calcaneus bone of the user's foot, scaling the intensity of the second stimulus based on a sensitivity of the hallux of the user's foot, scaling the intensity of the third stimulus based on a sensitivity of the cuboid of the user's foot, and scaling the intensity of the fourth stimulus based on a sensitivity of the medial aspect of the user's foot.

In any of the embodiments disclosed herein, the method can further include obtaining the respective sensitivities of the calcaneus bone, hallux, cuboid, and medial aspect of the user during a calibration.

In any of the embodiments disclosed herein, the one or more stimuli can include auditory feedback.

In any of the embodiments disclosed herein, the one or more stimuli can include visual feedback.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1A provides a perspective view of a haptic feedback device fitted on a user, in accordance with an exemplary embodiment of the present invention.

FIG. 1B provides a detail view of a vibrotactile cell of the haptic feedback device of FIG. 1A.

FIG. 1C provides a detail view of a microcontroller of the haptic feedback device of FIG. 1A.

FIG. 2A provides a perspective view of a shoe insert of a haptic feedback device, in accordance with an exemplary embodiment of the present invention.

FIG. 2B provides a detail view of a sensor of the shoe insert of FIG. 2A.

FIG. 3A provides a plot showing haptic plantar pressure modulation capability of a haptic feedback device, in accordance with an exemplary embodiment of the present invention.

FIG. 3B provides a plot showing haptic sensitivity on areas of the thigh and calf, in accordance with an exemplary embodiment of the present invention.

FIG. 4A provides a flowchart of a method of providing haptic feedback to a user, in accordance with an exemplary embodiment of the present invention.

FIG. 4B provides a flowchart of a method of providing haptic feedback to a user, in accordance with an exemplary embodiment of the present invention.

FIG. 4C provides a flowchart of a method of providing haptic feedback to a user, in accordance with an exemplary embodiment of the present invention.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

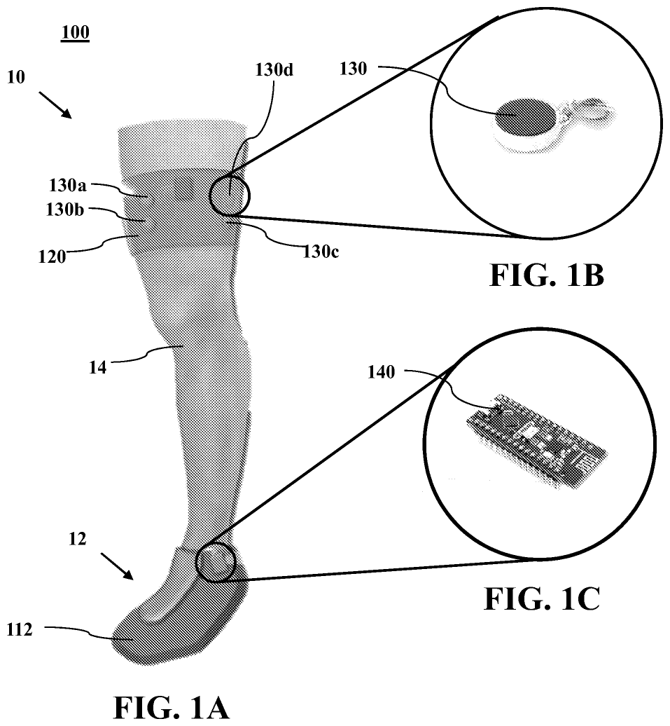

As shown in FIG. 1A, an exemplary embodiment of the present invention provides a haptic feedback device 100. Device 100 can include one or more pressure sensors 110 configured to measure a pressure applied to a foot 12 of a user 10 and a haptic feedback sleeve 120 configured to provide haptic feedback to the user 10. In some examples, the haptic feedback sleeve 120 can include one or more straps to secure the sleeve 120 to the user 10. The haptic feedback sleeve 120 can also include an elastic material configured to provide a compressive force to the user 10. The haptic feedback can be based, at least in part, on the pressure applied to the foot 12 of the user 10. The one or more pressure sensors 110 can be configured to measure pressure applied to a plantar aspect of the user's 10 foot 12. The one or more pressure sensors 110 are configured to measure pressure applied to one or more of the calcaneus bone, hallux, cuboid, and medial aspect of the user's 10 foot 12. The haptic feedback sleeve 120 shown in FIG. 1A is configured to be secured to a thigh of the user 10. In other examples, the haptic feedback sleeve 120 is configured to be secured to a calf of the user 10.

The haptic sleeve 120 can include one or more vibrotactile cells 130 (shown in the detail view in FIG. 1B) configured to apply a stimulus to the user 10. The one or more vibrotactile cells 130 are configured to apply a stimulus to at least a portion of a leg 14 of the user 10. The one or more vibrotactile cells 130 can include one or more of a first vibrotactile cell 130a configured to apply a stimulus to an anterior aspect of a leg 14 of the user 10, a second vibrotactile cell 130b configured to apply a stimulus to a posterior aspect of a leg 14 of the user 10, a third vibrotactile cell 130c configured to apply a stimulus to a medial aspect of a leg 14 of the user 10, and a fourth vibrotactile cell 130d configured to apply a stimulus to a lateral aspect of a leg 14 of the user 10. In some examples, two or more of the one or more vibrotactile cells 130 are configured to provide simultaneous stimuli to the user 10. Furthermore, two or more of the one or more vibrotactile cells 130 can be configured to provide sequential stimuli to the user 10.

FIG. 1C shows one or more microcontrollers 140 configured to collect measurements taken by the one or more pressure sensors 110 and control the haptic feedback provided to the user 10. The one or more microcontrollers 140 can be further configured to control one or more of a frequency, amplitude, or pattern of the haptic feedback provided to the user 10. The device 100 can further include an accelerometer configured to determine when the user 10 is moving. Accelerometer can in some examples be integrated into microcontroller 140.

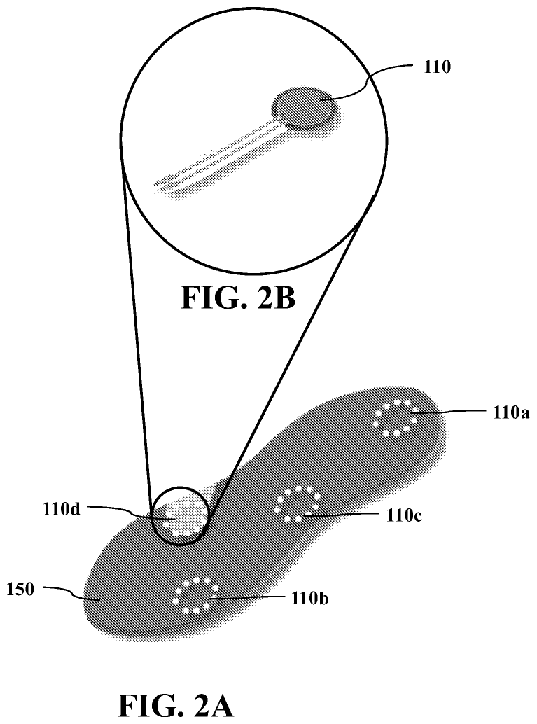

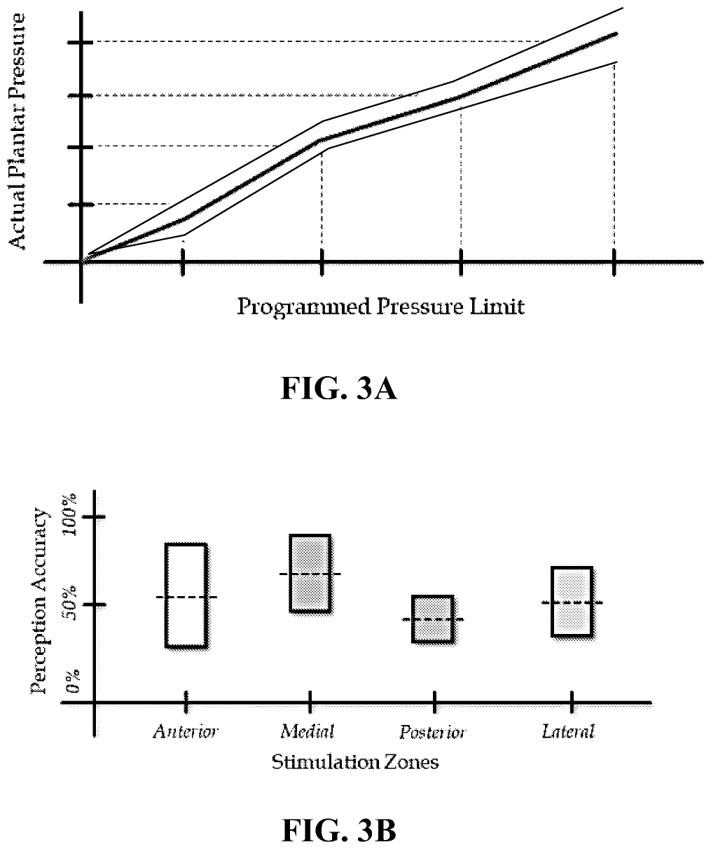

As shown in FIG. 2A, the one or more pressure sensors 110 can include one or more of a first pressure sensor 110a configured to measure pressure applied to a calcaneus bone of the user's 10 foot 12, a second pressure sensor 110b configured to measure pressure applied to a hallux of the user's 10 foot 12, a third pressure sensor 110c configured to measure pressure applied to a cuboid of the user's 10 foot 12, and a fourth pressure sensor 110d configured to measure pressure applied to a medial aspect of the user's 10 foot 12. As seen in the magnified view shown in FIG. 2B, the one or more pressure sensors 110 can be force sensitive resistors. As seen in FIG. 2A, the one or more pressure sensors 110 can be positioned on a shoe insert 150. Alternatively, the one or more pressure sensors 110 can be positioned in an injury/cast boot. In some examples, the haptic feedback is further based, at least in part, on a predetermined pressure threshold. The one or more pressure sensors 110 can be configured to wirelessly transmit pressure measurements to the haptic feedback sleeve 120.

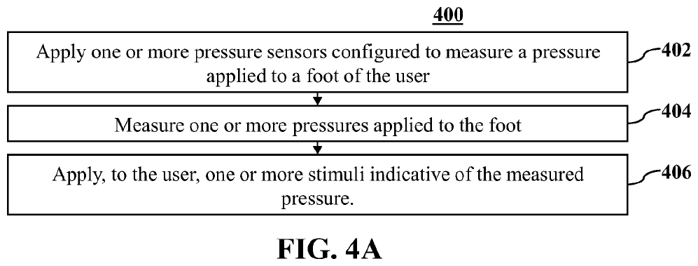

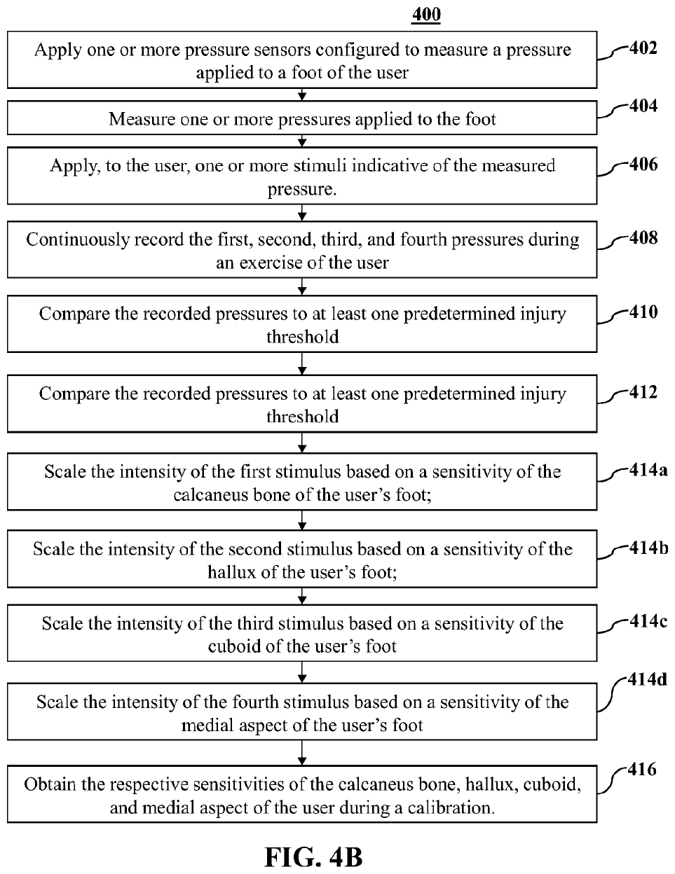

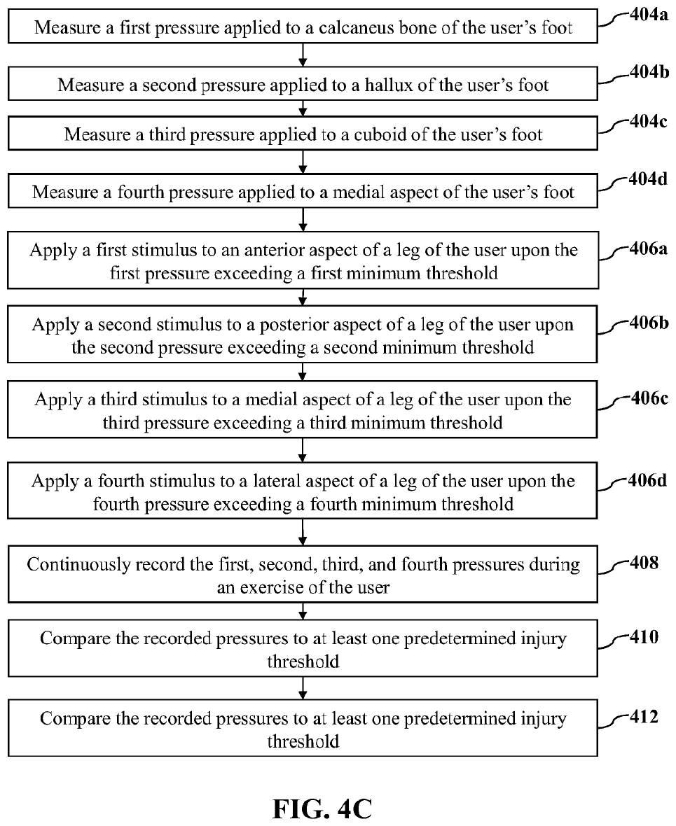

FIG. 4A shows a method 400 of providing haptic feedback to a user. The method 400 can include applying 402 one or more pressure sensors configured to measure a pressure applied to a foot of the user, measuring 404 one or more pressures applied to the foot, and applying 406, to the user, one or more stimuli based, at least in part, on the measured pressure.

As shown in FIGS. 4B-4C, measuring one or more pressures applied to the foot can include one or more of measuring 404a a first pressure applied to a calcaneus bone of the user's foot, measuring 404b a second pressure applied to a hallux of the user's foot, measuring 404c a third pressure applied to a cuboid of the user's foot, and measuring 404d a fourth pressure applied to a medial aspect of the user's foot. Applying 406 to the user one or more stimuli can include one or more of applying 406a a first stimulus to an anterior aspect of a leg of the user upon the first pressure exceeding a first minimum threshold, applying 406b a second stimulus to a posterior aspect of a leg of the user upon the second pressure exceeding a second minimum threshold, applying 406c a third stimulus to a medial aspect of a leg of the user upon the third pressure exceeding a third minimum threshold, and applying 406d a fourth stimulus to a lateral aspect of a leg of the user upon the fourth pressure exceeding a fourth minimum threshold.

In some examples, an intensity of one or more of the first, second, third, and fourth stimuli varies proportionally to the first, second, third, and fourth pressures, respectively.

Method 400 can further include continuously recording 408 one or more of the first, second, third, and fourth pressures during an exercise of the user, comparing 410 the one or more recorded pressures to at least one predetermined injury threshold, and increasing 412 the intensity of the one or more of the first, second, third, and fourth stimuli when the recorded pressures exceed the at least one predetermined injury threshold. The one or more stimuli can include vibrotactile feedback.

Method 400 can further include one or more of scaling 414a the intensity of the first stimulus based on a sensitivity of the calcaneus bone of the user's foot, scaling 414b the intensity of the second stimulus based on a sensitivity of the hallux of the user's foot, scaling 414c the intensity of the third stimulus based on a sensitivity of the cuboid of the user's foot, and scaling 414d the intensity of the fourth stimulus based on a sensitivity of the medial aspect of the user's foot. Method 400 can further include obtaining 416 one or more of the respective sensitivities of the calcaneus bone, hallux, cuboid, and medial aspect of the user during a calibration. In some embodiments, the one or more stimuli can include auditory feedback. In some embodiments, the one or more stimuli can include visual feedback.

The following examples further illustrate aspects of the present disclosure. However, they are in no way a limitation of the teachings or disclosure of the present disclosure as set forth herein.

### EXAMPLES

These examples describe a haptically-enabled foot ankle orthosis, such as the one presented above, including at least two components: a sensory feedback device and a pressure sensor-embedded shoe insert experiments to characterize and improve the function thereof. The haptically-enabled foot ankle orthosis is able to give vibrotactile feedback in the leg (from the sensory feedback device) in response to different stimuli sensed from the pressure sensor-embedded shoe insert. The study tests the ability of subjects to accurately interpret the mechanical feedback as a sensory replacement. The results from the study will inform us of the effectiveness of the device and guide any changes or improvements necessary.

To determine the efficacy of the haptically-enabled foot ankle orthosis at communicating sensory information to a sensing part of the body, in this study, human subjects with normal and healthy limbs wear a sensory feedback device around their leg which consists of a fabric cuff with embedded actuators that apply a vibrotactile stimulus to the participant. The study assesses the participant's ability and effort needed to distinguish the stimulus displayed by the cuff. In addition, a pressure sensor-embedded shoe insert is placed in the subject's respective “fictively injured” leg in order to monitor and modulate the amount of weight the subject has applied to their ‘fictively injured’ leg.

Human subjects are split into three sections: Stimulus Sensitivity Testing, Plantar Pressure Sensor Threshold Testing and Efficacy of Haptically-Enabled Foot Ankle Orthosis. Performance data will be collected throughout the experiment.

Relating to Stimulus Sensitivity Testing-the subject is seated, and the sensory feedback device is placed around their leg and fitted. The researcher demonstrates the operation of the sensory display, introducing and pairing each stimulus to its respective actuator. The researcher runs the participant through a brief version of the experiment by applying stimulus and asks the subject to guess which actuator the stimulus corresponds to. The researcher tells the subject whether their guess was or was not correct. Following this introduction, the subject is asked if they wish to continue with the study.

To ensure a proper fit and regularity among subjects, the tightness of the leg strap is measured using force sensitive resistors (FSR). Four FSR will be placed around the inner side of the strap. The band is tightened until the average reading of the FSR reaches the desired threshold. The band is also attached to the subject using skin tape in order to avoid shifting.

Relating to Human Subject Experiments: this experimental procedure is divided into three phases. The first one assesses the ability to feel and distinguish individual vibrotactile feedback, the second one measures the effectiveness against simultaneous vibrotactile stimuli, and the third measures the sensitivity threshold for vibrotactile stimuli.

In order to determine bilateral sensitivity, each subject will complete the test twice: 1) Right Leg, 2) Left Leg. For each phase of the experiment two locations on the leg will be tested, the calf and the thigh. The experiment is repeated twice using two different strap models made out of different materials, neoprene, and elastic nylon. The motors are placed on the subject's skin and is secured in place using skin tape. Each motor is always be placed in the same location. Finally, the motors are covered using the strap which is tightened using the FSR readings.

To test Individual Stimulus Distinguishability: first, the researcher tests the subject's ability to distinguish and identify the stimulus of individual actuators. The researcher activates each motor following a predetermined order to keep the procedure constant among participants. Each vibration motor is activated for a few seconds, the subject has to tell whether they felt the stimulus and is asked to identify the actuator providing stimulus. Then the stimulus is removed. The participant is not told whether their answers are correct or not. The participant may answer that the stimulus is unclear. After the completion of this task, the subject is asked to complete the NASA Task Load Index, Paas Scale, and SUS Questionnaire self-assessment surveys.

Relating to Simultaneous Stimuli Distinguishability: this round follows the same procedure as the previous one, except the participant is exposed to more than one stimulus at a time. Similarly, they are asked to point out the motors being activated each time. The participant is not told whether their answers are correct or not. They may answer that the stimulus is unclear. After the completion of this task, the subject is asked to complete self-assessment surveys.

Stimulus Level Distinguishability: for the final phase of the experiment only one of the actuators is activated each time. Throughout the experiment, the subject is presented with a vibrotactile stimulus and is asked to tell as soon as they perceive the stimulus. The researcher documents the intensity at which they felt the stimulus. The rest of the motors are actuated following the same procedure and the response is recorded. After the completion of this task, the subject is asked to complete self-assessment surveys.

### Relating to Plantar Pressure Sensor Threshold Testing

Experimental Preparation and Learning Period: The subject stands on a pressure pad, in order to determine the location of the four plantar sensors (Medial and Lateral Calcaneus, 4^{th} and 5^{th} Metatarsal Head, 1^{st} Metatarsal Head, Hallux). The researcher demonstrates the operation of the device. The researcher runs the participant through a brief version of the experiment by applying pressure to the shoe insert. Following this introduction, the subject is asked if they wish to continue with the study.

Planar Pressure Sensor Shoe Insert: the researcher places the pressure sensor shoe insert into the subject's shoe and places the battery pack around the subject's body. The subject stands on a force plate, and the researcher will records the subject's body weight. Then, the subject only places their foot with the device on the force plate and be told to apply weight based on varying percentages of their body weight. The % body weight on the force plate is compared to the values from the pressure sensor shoe insert. These values are used to ensure that the FSRs are properly calibrated to the subject.

Human Subject Experiments: Subject is told to complete the following movements in a non-weight bearing scenario (Standing with crutches, walking with crutches, sitting with feet flat, sitting with foot out and pressure on the heel). The researcher records FSR and accelerometer values. These values are analyzed in order to prevent false measuring.

Data Analysis and Conclusions—the Planar Pressure Sensor Threshold Testing results determine the minimum threshold value of pressure a subject can apply to their foot during a non-weight bearing session. In addition, the accelerometer readings determines when the subject is at rest, in order to prevent false readings. Using the data, the threshold values are used for the Efficacy of Haptically-Enabled Foot Ankle Orthosis testing.

### Relating to the Efficacy of Haptically-Enabled Foot Ankle Orthosis

Experimental Preparation and Learning Period: The subject use crutches to simulate a “fictively injured” leg. The researcher runs the participant through a brief version of the experiment by teaching the subject the standard 3-point crutch stance. The subject then proceeds to go through the experimental preparation and learning period as the Stimulus Sensitivity Testing and Plantar Pressure Sensor Threshold Testing as listed above.

Human Subject Experiments: The experimental procedure is divided into three scenarios in order to determine the most effective time period. Once the most effective time period is chosen, the experimental procedure will be completed with additional subjects for that one specific scenario.

The Short-Term experimental procedure consists of a 2-hour session where the subject walks around a terrain park.

The Half-Day experimental procedure consists of a total of 8 hours. The researchers spend an hour with the subjects after the learning period. After completing the short study, the subject continues using the device in their daily life. The subject must complete 4 additional hours with the device. The subject returns the sensory feedback device after they complete the 4 hours or 8 hours after the start of the study.

The Full-Day experimental procedure consists of a total of 24 hours. After completing the short study, the subject continues using the device in their daily life. The subject must complete 8 additional hours with the device. The subject returns the sensory feedback device after they complete the 8 hours or 24 hours after the start of the study.

The subjects are randomized into two groups: No feedback (Control Group) and with Feedback (Intervention Group). The subjects will not know which group they are in.

This experimental procedure is divided into two phases. The subject follows a path within the terrain park as the following test is completed. In order to determine bilateral sensitivity, each subject completes the test twice, where the first trial is when the right leg is ‘fictively injured’, and the second trial is when the left leg is ‘fictively injured’.

Effects of Vibrotactile Stimuli: The subject starts by walking with crutches in order to calibrate the sensory feedback device which is attached to their leg. While the subject is walking with the crutches, the sensory feedback device records the number of weight-bearing accidents at a threshold value as per the results from the Plantar Pressure Sensor Threshold test. After completion of this task, the subjects are asked to fill out self-assessment surveys.

Effects of Various Sensitivity Thresholds: This round follows the same procedure as the previous one, except the sensitivity weight-bearing on the pressure-sensor embedded shoe insert varies. The researcher documents the sensitivity during each phase and record the number of weight-bearing accidents during each phase. After the completion of this task, the subject is asked to complete self-assessment surveys.

Data Analysis and Conclusions—The Efficacy of Haptically-Enabled Foot Ankle Orthosis results are analyzed to determine how effective the device was to prevent the subjects from applying a load to their “fictively injured” leg. Based on the data gathered from these experiments, adjustments are made to the feedback device as needed to develop a more effective device.

Sensory Feedback Device: Throughout the study, a device designed to provide vibrotactile feedback is placed on the subject's leg in the form of a fabric adjustable cuff. The cuff will include four vibration motors that will provide feedback in the form of vibrotactile stimuli. The device does not collect data but will provide tactile stimulus to the leg which is monitored to assess its efficiency.

Force Sensitive Resistors (FSR): A total of eight FSRs is used for this study. Four FSRs are placed on the subject's leg to measure the tightness of the device. Four sensors are placed in the pressure sensor embedded shoe insert to measure the amount of weight the subject is applying to their foot.

Accelerometer: An accelerometer is placed in the pressure sensor embedded shoe insert to measure if the subject is moving or not.

Stimulus Distinguishability Test: The subjects are asked whether they can feel the stimuli and if so to identify which motor or motors are being actuated. The results are used to determine the device's effectiveness. The subject is asked to tell when they start feeling the stimuli. The results are used to determine the device's effectiveness and the sensitivity threshold.

Plantar Pressure Sensor Threshold Test: The subject is asked to control the weight-bearing on their weight bearing on their ‘fictively injured’ leg. The results determine the minimum threshold value of the FSRs in a non-weight bearing situation.

Efficacy of Haptically-Enabled Foot Ankle Orthosis test: The subjects are asked to control their weight bearing on their ‘fictively injured’ leg. The results determine the efficacy of the device to determine if the device can prevent subjects from applying various loads to their ‘fictively injured’ leg.

NASA Task Load Index survey: The NASA Task Load Index is a subjective survey for measuring mental workload by accessing performance in six categories. The subject responds to this survey after each task.

Paas Mental Effort Scale: The Paas Mental Effort scale is a nine-point self-assessment survey that subjectively determines the cognitive effort of the task at hand. The subjects are asked to complete this survey after the task, in order to determine their level of mental effort while detecting vibration feedback and applying varying plantar pressure during the study.

This study helps the investigators determine how effective the haptically-enabled foot ankle orthosis device provides vibrotactile sensory feedback in order to modulate the subject's weight bearing and plantar pressure. This device gives sensory feedback to patients suffering from ankle sprains and can assist in correcting gait abnormalities. The development of this technology is useful for patients recovering from injuries, as the vibrotactile feedback allows them to be more aware and in control of their walking. It also solves one of the major shortcomings of athletic training and sports injury prevention as the device can train athletes to modify their foot planting techniques to reduce the likelihood/severity of ankle sprains.

The Stimulus Sensitivity Testing results are analyzed to determine how effective the device is in displaying sensory feedback based on how accurately the subjects are able to distinguish the different stimuli in each task. The mental workload involved in identifying stimulus are also considered to determine how difficult it is to perceive and interpret the feedback stimulus.

The Plantar Pressure Sensor Threshold Test results are analyzed to determine the minimum threshold value for a non-weight bearing scenario.

The Efficacy of Haptically-Enabled Foot Ankle Orthosis results are analyzed to determine if the subjects are able to modify the amount of weight applied and the distribution of plantar pressures on their “fictively injured” leg.

This device is intended to be used to provide a means for orthopedic surgeons and their patients to monitor and modulate plantar pressures for recovery from acute ankle sprains. After surgery for acute ankle sprains, patients must avoid bearing weight on the affected foot until the joint is stable. Then, the patient gradually increases the weight on the foot until they reach normal, healthy load-bearing level. There are no currently available devices for this purpose.

Rechargeable AA batteries can be used to power the device. When pressure is applied pressure sensor shoe insert, data from each FSR is sent to the microcontroller. The accelerometer data is sent to the microcontroller for continuous monitoring. If the pressure in any of the FSRs exceeds the pressure threshold, and the accelerometer indicates that the subject is moving, data is sent to the corresponding actuator in the leg cuff to vibrate. Data from the FSRs, accelerometers, and the actuation level is stored for analysis.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

1. A haptic system comprising:
a wireless, pressure sensor-embedded shoe insert; and
a haptic-enabled feedback sleeve configured to provide haptic feedback to a user;
wherein:
the haptic feedback including cutaneous vibratory stimuli is based, at least in part, on feedback from the shoe insert; and
the system enables real-time monitoring and haptic modulation of dynamic pressures on the foot of the user

2. The haptic system of claim 1, wherein:
the haptic feedback is based, at least in part, on haptic plantar pressure feedback from the shoe insert;
the system enables real-time monitoring and haptic modulation of dynamic pressures on the plantar surface of the foot of the user; and
the shoe insert comprises one or more pressure sensors are configured to measure pressure applied to a plantar aspect of the user's foot.

3. The haptic system of claim 2, wherein one or more of the pressure sensors is configured to measure pressure applied to one or more of the calcaneus bone, hallux, cuboid, and medial aspect of the user's foot.

4. The haptic system of claim 2, wherein the one or more pressure sensors comprises:
a first pressure sensor configured to measure pressure applied to a calcaneus bone of the user's foot;
a second pressure sensor configured to measure pressure applied to a hallux of the user's foot;
a third pressure sensor configured to measure pressure applied to a cuboid of the user's foot; and
a fourth pressure sensor configured to measure pressure applied to a medial aspect of the user's foot.

5. The haptic system of claim 2, wherein the one or more pressure sensors are force sensitive resistors.

6. The haptic system of claim 2, wherein the haptic-enabled sleeve comprises:
an elastic material configured to provide a compressive force to the user; and
one or more vibrotactile cells configured to apply the cutaneous vibratory stimuli to the user.

7. The haptic system of claim 6, wherein the one or more vibrotactile cells are configured to apply the stimuli to at least a portion of a leg of the user.

8. The haptic system of claim 6, wherein the one or more vibrotactile cells comprises:
a first vibrotactile cell configured to apply a stimulus to an anterior aspect of a leg of the user;
a second vibrotactile cell configured to apply a stimulus to a posterior aspect of a leg of the user;
a third vibrotactile cell configured to apply a stimulus to a medial aspect of a leg of the user; and
a fourth vibrotactile cell configured to apply a stimulus to a lateral aspect of a leg of the user.

9. The haptic system of claim 6, wherein two or more of the one or more vibrotactile cells are configured to provide simultaneous stimuli to the user.

10. The haptic system of claim 6, wherein two or more of the one or more vibrotactile cells are configured to provide sequential stimuli to the user.

11. The haptic system of claim 6 further comprising:
one or more microcontrollers configured to receive signals from one or more of the pressure sensors and control the haptic feedback provided to the user; and
an accelerometer configured to determine when the user is moving.

12. The haptic system of claim 11, wherein the one or more microcontrollers are further configured to control one or more of a frequency, amplitude, or pattern of the haptic feedback provided to the user.

13. The haptic system of claim 11, wherein the haptic feedback is further based, at least in part, on a predetermined pressure threshold.

14. (canceled)

15. The haptic system of claim 11, wherein the haptic feedback sleeve further comprises one or more straps to secure the sleeve to the user.

16. (canceled)

17. The haptic system of claim 11, wherein the haptic feedback sleeve is configured to be secured to a calf of the user.

18. The haptic system of claim 11, wherein the haptic feedback sleeve is configured to be secured to a thigh of the user.

19. (canceled)

20. The haptic system of claim 11, wherein the shoe insert is positioned in an injury/cast boot.

21. (canceled)

22. A method comprising:
monitoring, in real-time, first dynamic pressure distributions on a weight-bearing foot of a user during locomotion;
providing feedback to the user in a location distal the foot based, at least in part, on the monitoring; and
applying, by the user, second dynamic pressure distributions on the weight-bearing foot different from the first dynamic pressure distributions based, at least in part, on the feedback.

23. The method of claim 22, wherein the monitoring comprises:
measuring a first pressure applied to a calcaneus bone of the user's foot;
measuring a second pressure applied to a hallux of the user's foot;
measuring a third pressure applied to a cuboid of the user's foot; and
measuring a fourth pressure applied to a medial aspect of the user's foot.

24. The method of claim 23, wherein the providing feedback comprises:
applying a first stimulus to an anterior aspect of a leg of the user upon the first pressure exceeding a first minimum threshold;
applying a second stimulus to a posterior aspect of a leg of the user upon the second pressure exceeding a second minimum threshold;
applying a third stimulus to a medial aspect of a leg of the user upon the third pressure exceeding a third minimum threshold; and
applying a fourth stimulus to a lateral aspect of a leg of the user upon the fourth pressure exceeding a fourth minimum threshold.

25. The method of claim 24, wherein an intensity of each of the first, second, third, and fourth stimuli varies proportionally to the first, second, third, and fourth pressures, respectively.

26. The method of claim 25 further comprising:
continuously recording the first, second, third, and fourth pressures during an exercise of the user;
comparing the recorded pressures to at least one predetermined injury threshold; and
increasing the intensity of each of the first, second, third, and fourth stimuli when the recorded pressures exceed the at least one predetermined injury threshold.

27. The method of claim 26, wherein the stimuli are selected from a group consisting of vibrotactile feedback, auditory feedback, visual feedback, and a combination thereof.

28. The method of claim 27 further comprising:
scaling the intensity of the first stimulus based on a sensitivity of the calcaneus bone of the user's foot;
scaling the intensity of the second stimulus based on a sensitivity of the hallux of the user's foot;
scaling the intensity of the third stimulus based on a sensitivity of the cuboid of the user's foot; and
scaling the intensity of the fourth stimulus based on a sensitivity of the medial aspect of the user's foot.

29.-31. (canceled)

32. The method of claim 27 further comprising:
obtaining acceleration data of the foot; and
adjusting the first, second, third, and fourth minimum thresholds based on the acceleration data.

33. The method of claim 22, wherein the applying comprises:
modulating magnitudes and distributions of pressures on the foot during locomotion.
